(19) United States
(12) Patent Application Publication
(10) Pub. No.: US 2017/0056176 A1
(43) Pub. Date: Mar. 2, 2017

(54) TREATMENTS FOR MITRAL VALVE INSUFFICIENCY

(71) Applicant: Edwards Lifesciences Corporation, Irvine, CA (US)

(72) Inventors: Stanton J. Rowe, Newport Coast, CA (US); Jinny Lee, Corona del Mar, CA (US); Philip P. Corso, JR., Laguna Hills, CA (US); Mark Chau, Aliso Viejo, CA (US); David L. Hauser, Newport Beach, CA (US)

(73) Assignee: Edwards Lifesciences Corporation, Irvine, CA (US)

(21) Appl. No.: 15/244,716

(22) Filed: Aug. 23, 2016

Related U.S. Application Data
(60) Provisional application No. 62/209,796, filed on Aug. 25, 2015.

(51) Int. Cl. A61F 2/24

(52) U.S. Cl. CPC A61F 2/2487; A61F 2/2418; A61F 2/2427; A61F 2/2466; A61F 2/2454; A61F 2/2463

(57) ABSTRACT

The present disclosure concerns embodiments of an implantable device that are used to treat an insufficient heart valve that has been previously treated by implantation of a fixation device or an Alfieri stitch that is secured to opposing portions of the native leaflets. In one representative embodiment, an implantable device for remodeling a native mitral valve having two native leaflets and a fixation device or an Alfieri stitch secured to respective free edges of the leaflets is configured to be coupled to the fixation device or Alfieri stitch and apply a remodeling force to the native mitral valve that draws the native leaflets toward each other to promote coaptation of the leaflets.

### CROSS REFERENCE TO RELATED APPLICATION

This application claims the benefit of U.S. Provisional Patent Application No. 62/209,796, filed Aug. 25, 2015, which is incorporated herein by reference.

### FIELD

This disclosure pertains generally to prosthetic devices and related methods for preventing or reducing regurgitation through native heart valves, as well as devices and related methods for implanting such prosthetic devices.

### BACKGROUND

The native heart valves (i.e., the aortic, pulmonary, tricuspid, and mitral valves) serve critical functions in assuring the forward flow of an adequate supply of blood through the cardiovascular system. These heart valves can be rendered less effective by congenital malformations, inflammatory processes, infectious conditions, or disease. Such damage to the valves can result in serious cardiovascular compromise or death. For many years the definitive treatment for such disorders was the surgical repair or replacement of the valve during open-heart surgery. However, such surgeries are highly invasive and are prone to many complications. Therefore, elderly and frail patients with defective heart valves often went untreated. More recently, transvascular techniques have been developed for introducing and implanting prosthetic devices in a manner that is much less invasive than open-heart surgery. Such transvascular techniques have increased in popularity due to their high success rates.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets extending downward from the annulus into the left ventricle. The mitral valve annulus can form a D-shaped, oval, or otherwise out-of-round cross-sectional shape having major and minor axes. The anterior leaflet can be larger than the posterior leaflet, forming a generally C-shaped boundary between the abutting free edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates, the oxygenated blood that is collected in the left atrium flows into the left ventricle. When the muscles of the left atrium relax and the muscles of the left ventricle contract, the increased blood pressure in the left ventricle urges the two leaflets of the mitral valve together, thereby closing the one-way mitral valve so that blood cannot flow back into the left atrium and is, instead, expelled out of the left ventricle through the aortic valve. To prevent the two leaflets from prolapse under pressure and folding back through the mitral valve annulus towards the left atrium, a plurality of fibrous cords called chordae tendineae tether the leaflets to papillary muscles in the left ventricle.

Mitral regurgitation occurs when the native mitral valve fails to close properly and blood flows into the left atrium from the left ventricle during the systole phase of the cardiac cycle. Mitral regurgitation is the most common form of valvular heart disease. Mitral regurgitation has different causes, such as leaflet prolapse, dysfunctional papillary muscles, and/or stretching of the mitral valve annulus resulting from dilation of the left ventricle. Mitral regurgitation at a central portion of the leaflets can be referred to as central jet mitral regurgitation, and mitral regurgitation nearer to one commissure (i.e., location where the leaflets meet) of the leaflets can be referred to as eccentric jet mitral regurgitation.

Some prior techniques for treating mitral regurgitation include stitching edge portions of the native mitral valve leaflets directly to one another (known as an Alfieri stitch). Other prior techniques include the implantation of a fixation member that mimics an Alfieri stitch by fixing edge portions of the native leaflets to one another. One commercially available fixation device is the Mitraclip®, available from Evalve, Inc. A substantial number of patients treated with an Alfieri stitch or a fixation member have experienced poor clinical outcome, that is, significant residual mitral regurgitation. In some cases, residual mitral regurgitation can be treated by implanting one or more additional fixation members or additional stitches. However, additional fixation members or stitches can increase the pressure gradient across the mitral to an unacceptable level. Thus, there exists a need for treating patients that experience mitral regurgitation after implantation of a fixation device or treatment with an Alfieri stitch.

### SUMMARY

The present disclosure concerns embodiments of an implantable device that are used to treat an insufficient heart valve that has been previously treated by implantation of a fixation device or an Alfieri stitch that is secured to opposing portions of the native leaflets. Such fixation devices or Alfieri stitches typically are implanted in the native mitral valve. Thus, embodiments disclosed herein are described in the context of treating a native mitral valve. However, it should be understood that any of the disclosed embodiments can be used to treat the other valves of the heart (the aortic, pulmonary, and tricuspid valves).

In one representative embodiment, an implantable device for remodeling a native mitral valve having two native leaflets and a fixation device or an Alfieri stitch secured to respective free edges of the leaflets is configured to be coupled to the fixation device or Alfieri stitch and apply a remodeling force to the native mitral valve that draws the native leaflets toward each other to promote coaptation of the leaflets.

In some embodiments, the remodeling force applied by the implantable device draws the leaflets and the chordae tendineae closer toward the left atrium. In certain embodiments, the implantable device comprises a tension member configured to be coupled to the fixation device or Alfieri stitch and an anchor member connected to the tension member. The tension member can comprise, for example, an elongated, flexible piece of material, such as a suture, string, cord, wire, or similar material. The anchor member can be configured to engage tissue in or adjacent the heart, such as tissue in the left atrium, the intra-atrial septum, and/or a pulmonary vein. In some embodiments, the anchor member can comprise an expandable stent sized to be implanted within a pulmonary vein, which can include an eyelet through which the tension member can extend. In other embodiments, the anchor member can comprise a first anchor portion and a second anchor portion, the first anchor portion being configured to engage the intra-atrial septum in the left atrium and the second anchor portion being configured to engage the intra-atrial septum in the right atrium.

In some embodiments, the remodeling force applied by the implantable device causes the leaflets to be twisted about an axis extending parallel to the flow of blood from the left atrium to the left ventricle. In certain embodiments, the implantable device can be configured to be anchored to tissue in the left ventricle or the left atrium.

In another representative embodiment, a method for treating a native mitral valve of a heart having two native leaflets and a fixation device or an Alfieri stitch secured to respective free edges of the leaflets comprises delivering a remodeling device into the heart, coupling the remodeling device to the fixation device or Alfieri stitch, and applying a remodeling force to the native mitral valve via the remodeling device, the remodeling force drawing the leaflets toward each other to promote coaptation of the leaflets.

In certain embodiments, the method further comprises anchoring an anchor member of the remodeling device to tissue in or adjacent the heart to maintain the remodeling force on the native mitral valve.

In certain embodiments, the remodeling device comprises a tension member that is coupled to the fixation device or Alfieri stitch and is held in tension by an anchor member of the remodeling device that is anchored to tissue in or adjacent the left atrium. In some embodiments, the tension member forms a loop around the fixation device or Alfieri stitch and has two ends connected to the anchor member.

In another representative embodiment, a method for treating a native mitral valve of a heart having two native leaflets and a fixation device or an Alfieri stitch secured to respective free edges of the leaflets comprises coupling a docking member to the fixation device or Alfieri stitch and deploying a prosthetic valve within the docking member. In some embodiments, the act of coupling a docking member to the fixation device or Alfieri stitch comprises deploying a rail around the fixation device or Alfieri stitch and advancing the docking member along the rail to a location adjacent the native mitral valve within the left atrium. In some embodiments, the docking member comprises a radially extending flange that forms a seal against the inner surface of the left atrium. In some embodiments, the prosthetic valve is delivered into the heart in a radially compressed state by a delivery catheter and then radially expanded to an expanded state within the docking member.

In another representative embodiment, a method for treating a native mitral valve of a heart having two native leaflets and a fixation device or an Alfieri stitch secured to the leaflets at a location between the commissures so as to define two orifices between the leaflets separated by the fixation device or Alfieri stitch comprises implanting a prosthetic valve within one of the orifices. In some embodiments, the method further comprises implanting another prosthetic valve in the other orifice. In some embodiments, the prosthetic valves are connected to each other by a connecting member.

In another representative embodiment, an assembly for treating a native mitral valve of a heart having two native leaflets and a fixation device or an Alfieri stitch secured to respective free edges of the leaflets comprises a docking member configured to be coupled to the fixation device or Alfieri stitch and a prosthetic valve configured to be deployed within the docking member.

The foregoing and other objects, features, and advantages of the invention will become more apparent from the following detailed description, which proceeds with reference to the accompanying figures.

### BRIEF DESCRIPTION OF THE DRAWINGS

FIG. 1 shows a cross-section of the heart and a fixation device secured to the native mitral valve leaflets.

FIG. 2 shows the application of a remodeling force to the native mitral valve of FIG. 1 to improve coaptation of the native mitral valve leaflets.

FIG. 3 is a top plan view of the native mitral valve of FIG. 1 showing a tension member extending around the fixation device and applying the remodeling force to the native mitral valve.

FIG. 4 is a cross-sectional view of the heart showing the tension member of FIG. 3 extending around the fixation device.

FIGS. 5-7 are cross-sectional views of a heart showing the implantation of a remodeling device to remodel a native mitral valve having a fixation device, according to one embodiment.

FIG. 8 is a cross-sectional view of a heart showing another embodiment of a remodeling device that remodels the native mitral valve.

FIG. 9 is a cross-sectional view of a heart showing another embodiment of a remodeling device that remodels the native mitral valve.

FIGS. 10 and 11 are cross-sectional views of a heart showing the implantation of a docking member above a native mitral valve having a fixation member, according to one embodiment.

FIG. 12 is a cross-sectional view of a heart showing the implantation of a prosthetic valve in the docking member of FIG. 11.

FIG. 13 is a cross-sectional view of a heart similar to FIG. 12 but showing an alternative embodiment of a docking member for a prosthetic valve.

FIG. 14 is a top plan view of a native mitral valve having a fixation device secured to the free edges of the native leaflets.

FIG. 15 is a top plan view of a native mitral valve similar to FIG. 14 but showing the application of a remodeling force in a rotational direction extending around an axis that extends parallel to the flow of blood from the left atrium to the left ventricle.

FIG. 16 is a cross-sectional view of a heart showing a remodeling device that applies a remodeling force to the native mitral valve in a rotational direction, according to another embodiment.

FIG. 17 is a cross-sectional view of a heart showing another embodiment of a remodeling device that applies a remodeling force to the native mitral valve in a rotational direction.

FIG. 18 is a cross-sectional view of a heart showing another embodiment of a remodeling device that applies a remodeling force to the native mitral valve in a rotational direction.

FIG. 19 shows a dual-prosthetic valve assembly implanted in a native mitral valve having a fixation device secured to the free edges of the native leaflets.

### DETAILED DESCRIPTION

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods. As used herein, the terms “a”, “an”, and “at least one” encompass one or more of the specified element. That is, if two of a particular element are present, one of these elements is also present and thus “an” element is present. The terms “a plurality” of and “plural” mean two or more of the specified element.

As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “coupled” generally means physically coupled or linked and does not exclude the presence of intermediate elements between the coupled items absent specific contrary language.

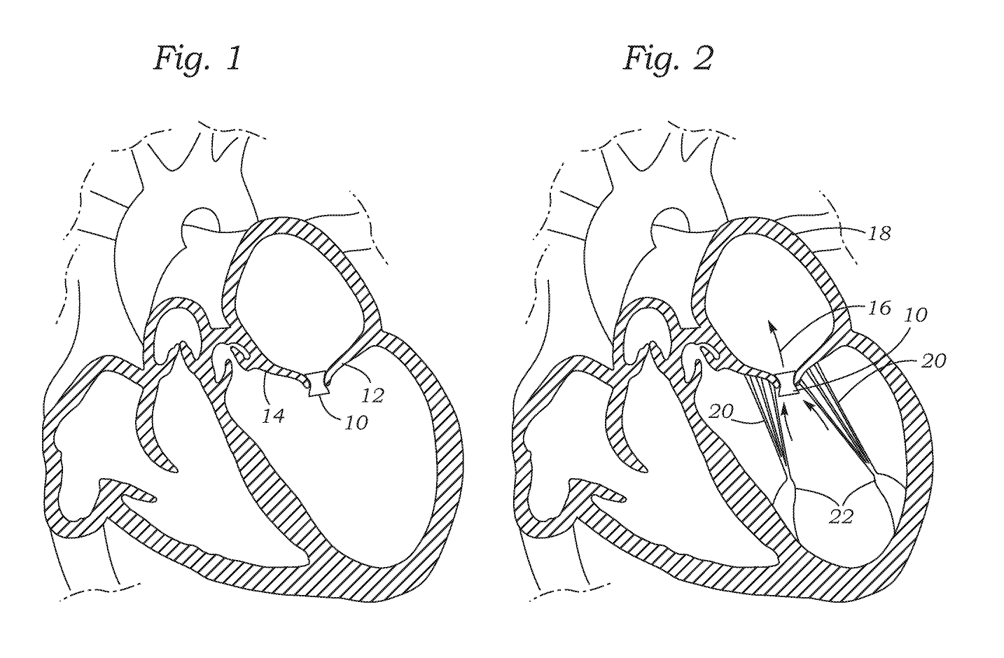

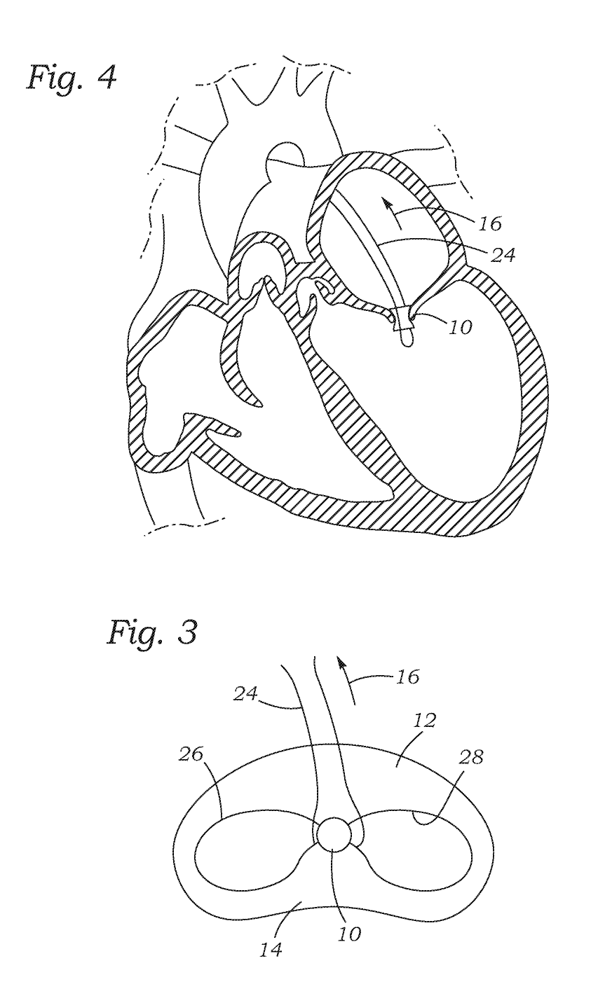

FIG. 1 depicts a known fixation device 10 secured to the native leaflets 12, 14 of the mitral valve. The fixation device 10 typically is secured to the center portion of the free edges of the native leaflets 12, 14, thereby defining two flow orifices 26, 28 on opposite sides of the fixation device (FIG. 3). The fixation device 10 functions to bring the free edges of the native leaflets closer together to promote coaptation and reduce mitral regurgitation. As explained above, in many patients the fixation device fails to reduce mitral regurgitation to an acceptable level. Although the figures show devices and methods for treating a native valve previously treated with a fixation device 10, it should be noted that the disclosed embodiments also can be used to treat a native valve previously treated with an Alfieri stitch securing the edges of the leaflets together. Thus, in the figures, reference number 10 also represents an Alfieri stitch.

FIG. 2 depicts the application of a remodeling force, indicated by arrow 16, to the native leaflets in a direction toward the left atrium 18 along an axis that is parallel or generally parallel to the flow of blood from the left atrium to the left ventricle. The remodeling force 16 remodels or reshapes native mitral valve by pulling the native leaflets 12, 14 inwardly toward each other and upwardly toward the left atrium, as well as the chordae tendineae 20 and the papillary muscles 22 inwardly toward each other and upwardly toward the left atrium 18 toward their natural position beneath the commissures of the leaflets 12, 14, thereby improving coaptation of the leaflets, and reducing or preventing mitral regurgitation.

FIGS. 3 and 4 depict an implantable remodeling or reshaping device in the form of a tension member 24 configured to apply a remodeling force 16 to native leaflets connected by a fixation device 10. In the illustrated embodiment, the tension member 24 forms a loop around the fixation device 10 and is pulled or tensioned upwardly to apply a remodeling force 16 to remodel the native leaflets, the chordae tendineae, and the papillary muscles. The tension member 24 comprises, for example, a thin, elongated and flexible material, such as a suture, string, chord, or a wire. A desired amount of tension in the tension member 24 can be retained by securing the upper ends of the tension member to an anchoring member deployed in or adjacent the heart, such as in the left atrium, the atrial septum, and/or a pulmonary vein, as further described below. The anchor member desirably maintains the remodeling force on the heart tissue and therefore maintains the heart tissue in a remodeled or reshaped state.

In another embodiment, the tension member 24 need not form a loop around the fixation device 10 and instead can have a first, lower end secured to the fixation device and an upper end secured to an anchor member deployed in or adjacent the heart, such as in the left atrium, the atrial septum, and/or a pulmonary vein.

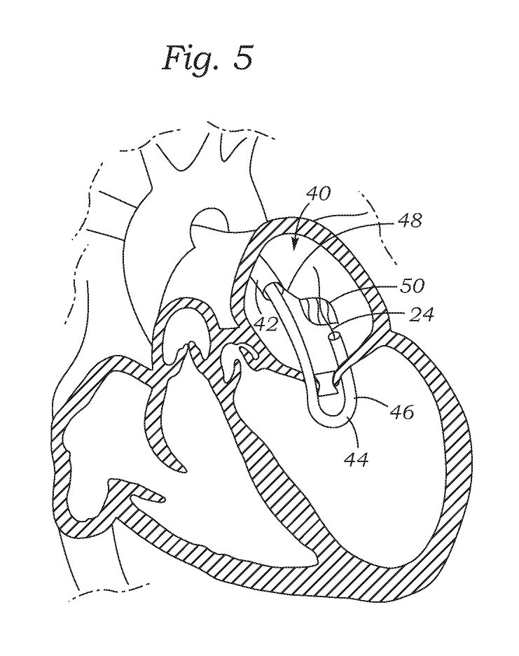

FIG. 5 depicts a delivery apparatus 40 and associated method for deploying a remodeling device within the heart. The delivery apparatus 40 in the illustrated embodiment comprises a trans-septal catheter 42, a deployment catheter 44, and a snare 48. In use, the trans-septal catheter 42 can be introduced into the body and advanced through the patient's vasculature to the heart in an antegrade approach. For example, the trans-septal catheter 42 can be inserted into a femoral vein and advanced through the inferior vena cava, into the right atrium of the heart, and pushed across the intra-atrial septum into the left atrium. In another approach, the trans-septal catheter 42 can be inserted into a jugular vein and advanced through the superior vena cava to the heart. The trans-septal catheter 42 can have a steering mechanism, such as one or more pull wires extending the length of the catheter, configured to adjust the curvature of the distal end portion of the catheter 42 to assist in steering the catheter through the patient's vasculature. Further details of the delivery apparatus are disclosed in U.S. Publication No. 2015/0230919, which is incorporated herein by reference.

The deployment catheter 44 can be advanced through a lumen of the trans-septal catheter 42 until a distal end portion 46 of the deployment catheter 44 extends outwardly from the distal end of the trans-septal catheter 42. The distal end portion 46 of the deployment catheter 44 desirably is configured to form a 180-degree curve or bend so that it can be placed to extend through orifices 26, 28 and around the fixation device 10, as shown in FIG. 5. The distal end portion 46 can be pre-formed (such as by heat shaping) to have a 180-degree curve in a non-deflected state. The pre-formed distal end portion 46 can be deflected to a non-curved, substantially straight configuration for advancement through the trans-septal catheter. When the distal end portion 46 is advanced from the distal opening of the trans-septal catheter, the distal end portion 46 can revert back to the non-deflected, curved configuration. Alternatively, the deployment catheter 44 can be provided with a steering mechanism, such as a pull wire, that is configured to bend the distal end portion from a straight configuration to the curved configuration shown in FIG. 5.

After the curved distal end portion 46 is placed around the fixation device 10, a tension member 24 can be advanced through and deployed from the distal end of the deployment catheter 44. The snare 48 can then be advanced from the trans-septal catheter 42 to capture and retract the tension member 24. A snare loop 50 of the snare 48 is placed around the distal end of the tension member and retracted back into the trans-septal catheter 42. The snare 48 can be retracted out of the proximal end of the trans-septal catheter 42 so that the opposing ends of the tension member 24 reside outside the body.

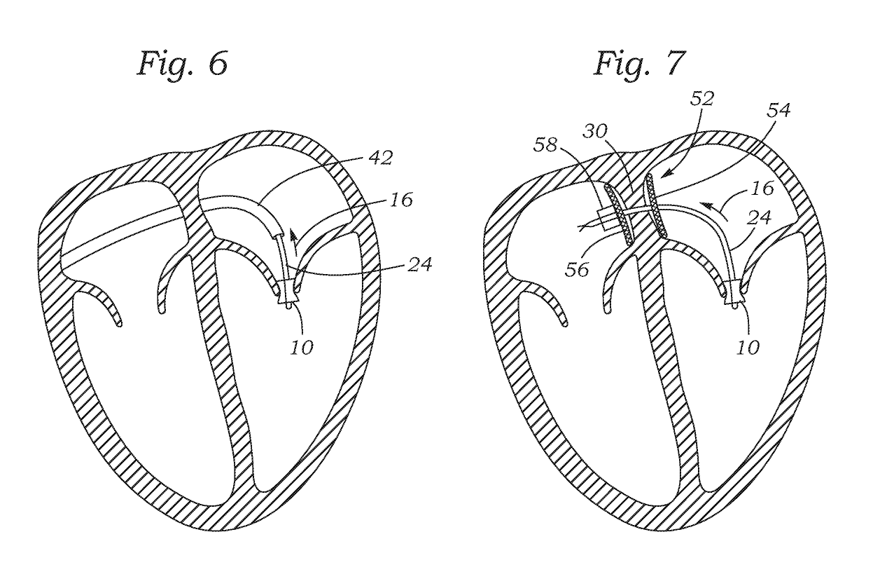

As shown in FIG. 6, the deployment catheter 44 can be withdrawn from the body, leaving the tension member 24 in place extending around the fixation device 10 and through the trans-septal catheter 42. A remodeling force 16 can be applied to the heart tissue by pulling the ends of the tension member 24 proximally to remodel the heart tissue, as explained above.

Referring to the FIG. 7, an anchor member 52 can be deployed from the trans-septal catheter 42 by advancing the anchor member 52 distally over the tension member 24 through the trans-septal catheter 42 and into the heart. The anchor member 52 in the illustrated embodiment comprises a first anchor portion 54 and a second anchor portion 56. The first anchor portion 54 can be deployed against the intra-atrial septum 30 in the left atrium and the second anchor portion 56 can be deployed against the septum 30 in the right atrium. The portions of the tension member 24 in the right atrium can be cut or severed and the severed end portions can be tied off to each other to prevent them from being pulled through the anchor portions 54, 56.

Alternatively, a fastener 58 (such as a suture clip) can be advanced over the tension member 24 and pushed against the second anchor portion 56 before severing the tension member 24. The fastener 58 can be a suture clip, or another type of fastener that can be deployed from a catheter and secured to a suture within the patient's body. Various suture clips and deployment techniques for suture clips that can be used in the methods disclosed in the present application are disclosed in U.S. Publication Nos. 2014/0031864 and 2008/0281356 and U.S. Pat. No. 7,628,797, which are incorporated herein by reference. In the case of a slidable fastener, the fastener 58 can be movable along the tension member 24 in a direction toward the septum, and configured to resist movement along the tension member in the opposite direction.

In particular embodiments, the deployment catheter 44, the snare 48, and the tension member 24 can be pre-loaded within the trans-septal catheter 42 and all components can be delivered into the left atrium together as a unit. Each component can then be advanced from the trans-septal catheter 42 in the sequence described above.

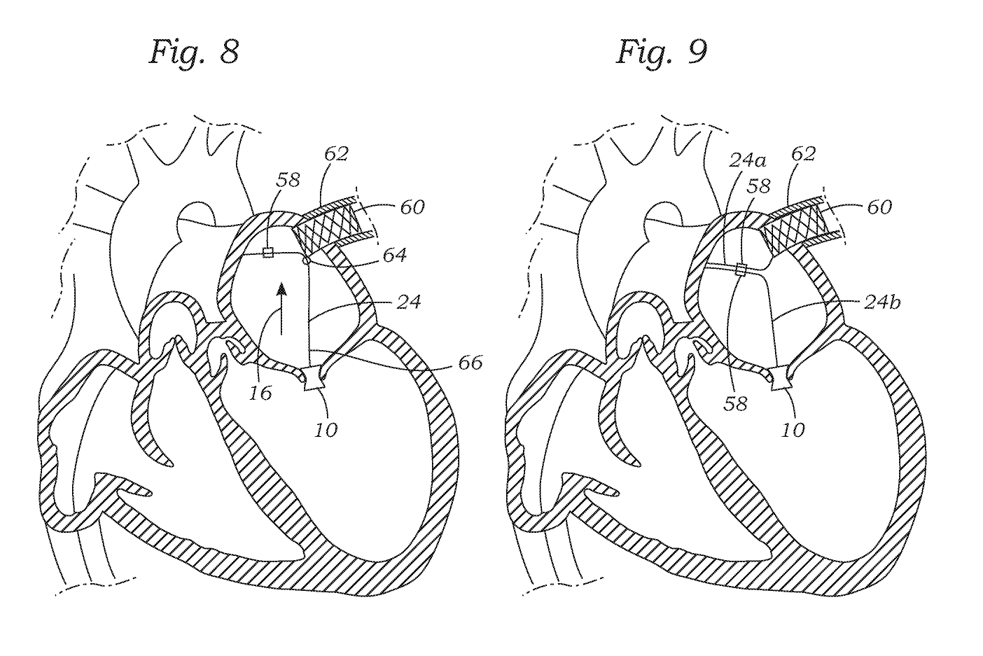

FIG. 8 shows the implantation of a remodeling device, according to another embodiment. In the embodiment of FIG. 8, the remodeling device comprises a tension member 24 and an anchor member in the form of an expandable stent 60 deployed within a pulmonary vein 62. The tension member extends through the stent 60 and has a distal end 66 secured to a fixation device 10. The stent 60 can have an eyelet 64 through which the tension member is threaded. The tension member 24 in this embodiment comprises a single length of the tension member rather than a loop extending around the fixation device 10. The distal end 66 of the tension member can be secured to a fastening member (not shown) that engages and secures the distal end 66 to the fixation device 10. In alternative embodiments, the tension member 24 can be looped around the fixation member, as described above, with both lengths of the tension member extending through the eyelet 64.

The stent 60 can be a self-expandable stent (made of a self-expandable material, such as Nitinol) or a plastically-expandable stent (made of a plastically expandable material, such as stainless steel or a cobalt-chromium alloy). In the case of a self-expandable stent, the stent can be delivered to the heart in a radially compressed state inside a sheath of a delivery catheter, as known in the art. The stent can be deployed from the sheath into the pulmonary vein, whereupon the stent can self-expand to a radially expanded state against the inner surface of the pulmonary vein. In the case of a plastically-expandable stent, the stent can be radially compressed on a balloon (or equivalent expansion mechanism) of a delivery catheter and advanced through the patient's vasculature into the pulmonary vein, whereupon the balloon can be inflated to expand the stent against the inner surface of the pulmonary vein.

After deploying the tension member 24 and the stent 60, the tension member 24 can be pulled proximally to apply a remodeling force 16 to remodel the heart tissue. A fastener 58 can then be advanced over the tension member 24 against the eyelet 64 to maintain tension on the tension member, after which the tension member can be severed proximal to the fastener. In particular embodiments, the stent 60 and the fastener 58 can be pre-loaded on the tension member 24 within the deployment catheter 44 (not shown in FIG. 8) with the stent 60 positioned distal to the fastener 58. After securing the tension member 24 to the fixation device, the stent 60 can be deployed within the pulmonary vein 62, followed by deployment of the fastener 58.

FIG. 9 shows the implantation of a remodeling device, according to another embodiment. In the embodiment of FIG. 9, the remodeling device comprises first and second tension members 24a, 24b, respectively, and an expandable stent 60 deployed within a pulmonary vein 62. The distal end of the first tension member 24a is secured to the stent 60 and the distal end of the second tension member 24b is secured to the fixation device 10. Both tension members 24a, 24b extend through a fastener 58, which can be advanced distally while pulling the tension members 24a, 24b proximally to apply a desired amount of remodeling force to the heart tissue. Thereafter, the tension members can be severed at a location proximal to the fastener 58.

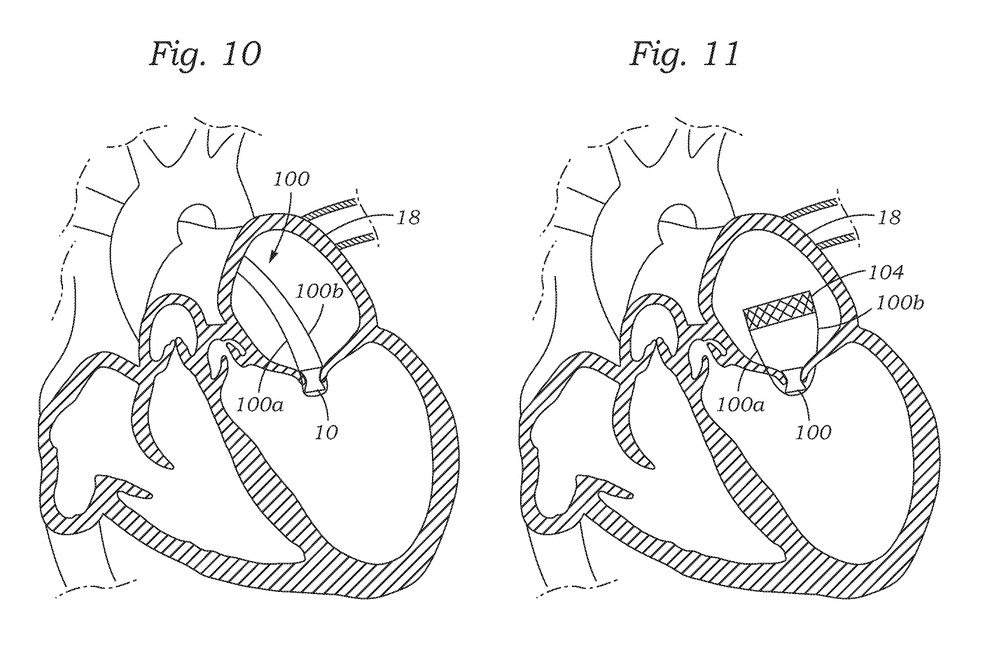

FIGS. 10-13 illustrate a procedure for implanting a prosthetic heart valve within the left atrium 18 utilizing a fixation device 10 as a support for the prosthetic valve. As shown in FIG. 10, a rail 100 can be positioned to extend through orifices 26, 28 and around the fixation device in the manner described above with respect to FIGS. 3-6, forming two side-by-side rail portions 100a, 100b extending upwardly from the fixation device 10. The delivery apparatus 40 (FIG. 5) can be used to deliver and position the rail as shown in FIG. 10. The rail 100 desirably comprises a metal wire or similar material that has sufficient flexibility to be looped around the fixation device yet has sufficient rigidity to support a prosthetic valve against the pressure gradient within the left atrium.

Referring now to FIG. 11, a docking member or docking ring 104 can be advanced distally over the rails portions 100a, 100b to a location adjacent the native mitral valve within the left atrium 18. The docking ring 104 can be a self-expandable stent (e.g., made of Nitinol) that can be delivered to the patient's heart in a radially compressed position within a sheath of a delivery catheter and can expand to a radially expanded state once deployed from the sheath. After positioning the rail 100 around the fixation device 10, the ends of the rail (which can be pulled outside of the body) can be threaded through respective openings or eyelets in the docking ring 104. The docking ring 104 can loaded into a delivery catheter in a radially compressed state and advanced over the rail portions 100a, 100b through the patient's vasculature. Once inside the left atrium, the docking ring 104 can be deployed from the catheter, allowing the docking ring to expand to the radially expanded state shown in FIG. 11.

Respective fasteners (not shown in FIG. 11), such as fasteners 58, can be deployed over the rail portions 100a, 100b proximal to the docking ring 104 to retain the docking ring on the rail portions. Alternatively, the docking ring 104 can have integral locking members or fasteners that can be activated to engage and secure the docking ring at a desired location along the rail portions 100a, 100b.

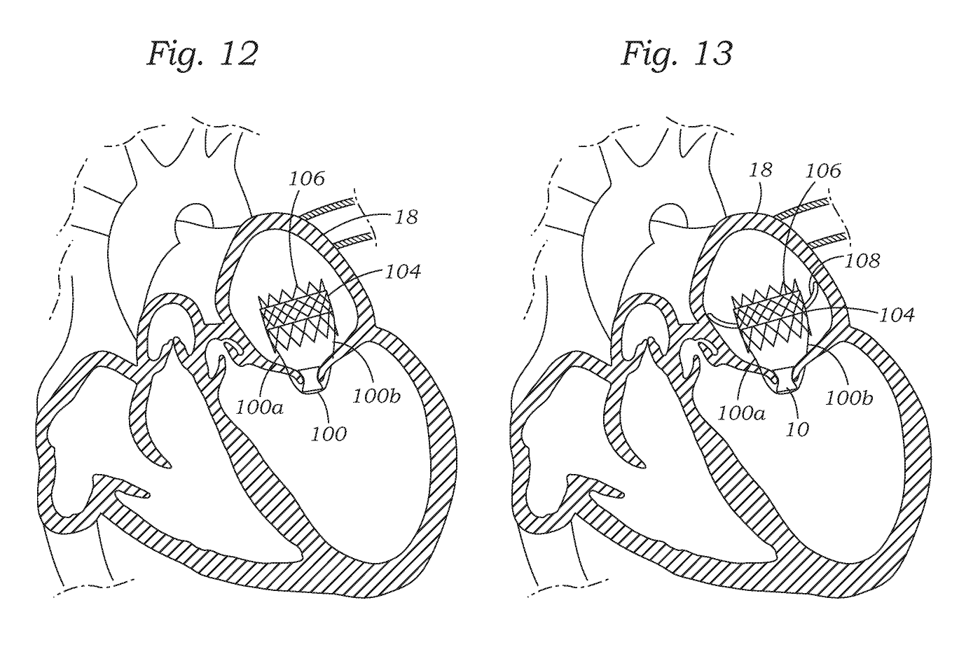

Referring now to FIG. 12, after deployment of the docking ring 104, a prosthetic heart valve 106 can be deployed within the docking ring 104. The prosthetic heart valve 106 can be an expandable, transcatheter heart valve. The prosthetic valve 106 can delivered and implanted with a separate delivery catheter that can be advanced through the patient's vasculature in a trans-septal delivery approach as described above. The prosthetic valve 106 can comprises a metal frame or stent that supports one or more prosthetic leaflets that regulate the flow of blood through the prosthetic valve, as known in the art. The prosthetic valve can be a self-expandable or plastically-expandable prosthetic valve. Some examples of prosthetic valves that can be used are disclosed in, for example, U.S. Pat. Nos. 7,993,394; 7,393,360; and 8,652,202, and U.S. Publication No. 2012/0123529, which are incorporated herein by reference. The prosthetic valve 106 can work in series with the native leaflets 12, 14 to help regulate the flow of blood between the left atrium and the left ventricle while minimizing or preventing mitral regurgitation.

FIG. 13 shows an embodiment similar to FIG. 12, except that the docking ring 104 is formed with an annular, radially extending flange 108 that can form a seal against the inner wall of the left atrium 18. In this manner, blood entering the left atrium from the pulmonary veins is directed to flow into the prosthetic valve 106 and then through the native leaflets 12, 14. The flange 108 can extend completely around the outer surface of the docking ring 104 (i.e., the flange can extend 360 degrees around the docking ring).

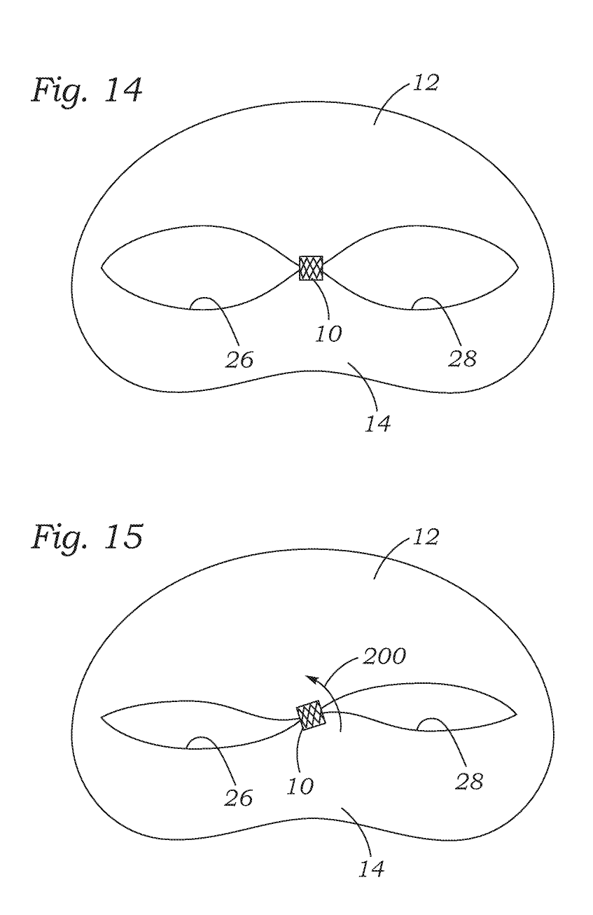

FIGS. 14 and 15 illustrate another procedure for treating mitral regurgitation in a patient previously treating with a fixation device 10. FIG. 14 is a top plan view of a mitral valve (as viewed from the left atrium) having a fixation device 10 holding the center edge portions of the leaflets 12, 14 together, forming orifices 26, 28 on opposite sides of the fixation device. In FIG. 15, the fixation device 10 is twisted or rotated in the direction of arrow 200 about an axis perpendicular to the page (i.e., an axis extending from the left atrium to the left ventricle parallel to the flow of blood). Twisting the fixation device 10 effectively reduces the size of the orifices 26 and 28 and brings the free edges of the native leaflets 12, 14 closer to each other, which in turn promotes coaptation of the leaflets and prevents or minimizes mitral regurgitation.

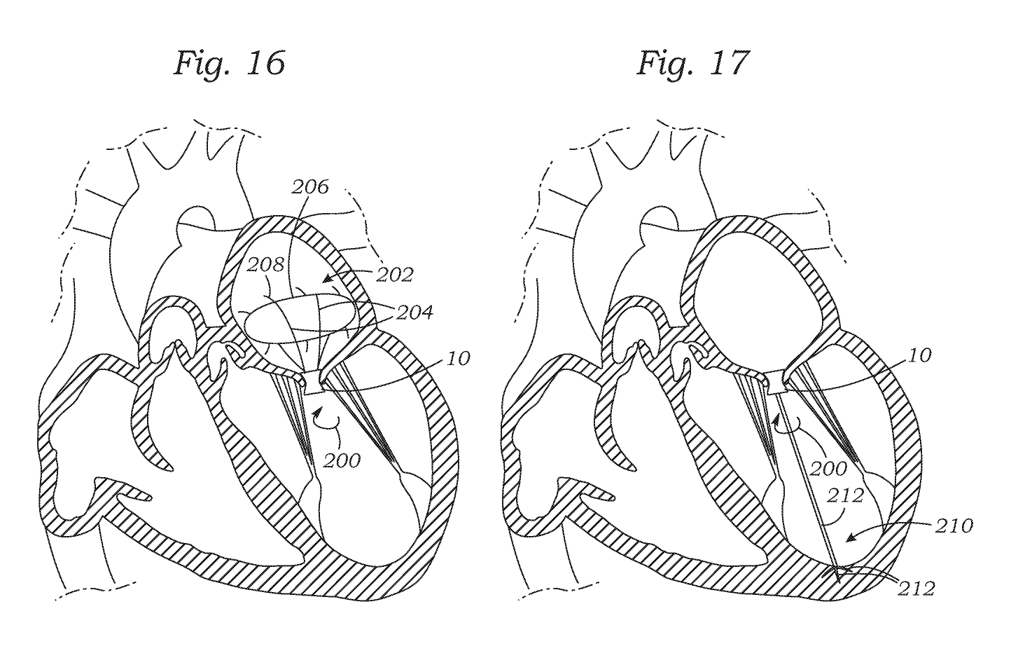

FIG. 16 shows a remodeling device 202, according to another embodiment, that can be used to apply and maintain a remodeling force 200 on the fixation device 10 and the native leaflets 12, 14. The remodeling device 202 comprises one or more legs or struts 204 that can be connected to the fixation device 10 at their lower ends and an anchoring ring 206 connected to the upper ends of the struts 204. The ring 206 can include a plurality of circumferentially spaced, curved barbs or hooks 208 that extend radially outwardly from the ring for engaging the inner wall of the left atrium.

In use, the remodeling device 202 can be delivered to the left atrium using a delivery catheter (not shown) and secured to the fixation device 10. While the remodeling device 202 is still connected to the delivery catheter, the delivery catheter can be rotated in the direction of arrow 200, which in turn rotates the remodeling device 202 and draws the native leaflets 12, 14 closer toward each as shown in FIG. 15. The remodeling device can then be disconnected from the delivery catheter. The barbs 208 are curved in the opposite direction of the rotation of the remodeling device 202. In this manner, the barbs 208 do not resist rotation of the remodeling device 202 when rotated to apply the remodeling force to the native leaflets, but can engage and/or penetrate adjacent tissue and resist rotation of the remodeling device in the opposite direction when the remodeling device is disconnected from the delivery catheter.

FIG. 17 shows a remodeling device 210, according to another embodiment, that can be used to apply and maintain a remodeling force 200 on the fixation device 10 and the native leaflets 12, 14. The remodeling device 210 comprises a shaft 212 and a plurality of tissue-engaging prongs or barbs 212 extending from the lower end of the shaft. The remodeling device 210 can be delivered to the heart (e.g., through a surgical opening in the left ventricle) and connected to the fixation device 10 at the upper end of the shaft 212. The remodeling device 210 can be rotated in the direction of arrow 200, after which the prongs 212 can be deployed into tissue in the left ventricle to resist rotation of the remodeling device in the opposite direction.

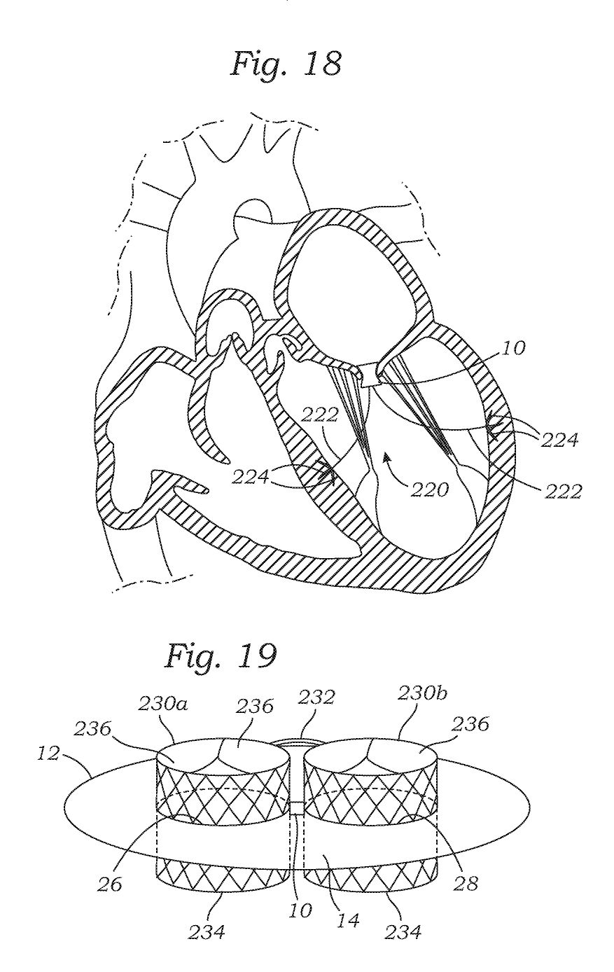

FIG. 18 shows a remodeling device 220, according to another embodiment, that can be used to apply and maintain a remodeling force 200 on the fixation device 10 and the native leaflets 12, 14. The remodeling device 220 can include a plurality of elongated struts 222, the lower ends of which include a plurality of prongs or barbs 224. The upper ends of the struts 222 can be connected to the fixation device 10, after which the remodeling device 220 can be rotated and held in place by prongs 224 embedded in tissue in the left ventricle.

FIG. 20 shows another technique that can be used to treat mitral deficiency. In this embodiment, a dual heart valve assembly comprising a first prosthetic heart valve 230a and a second prosthetic heart valve 230b are deployed within orifices 26 and 28, respectively, on opposite sides of a fixation device 10. The fixation device 10 serves as a base against which the prosthetic valves can be expanded. The prosthetic heart valves 230a, 230b can be connected to each other by one or more struts or connecting arms 232 to help stabilize the prosthetic valves and resist migration in at least one direction.

Each prosthetic valve 230a, 230b can comprise a radially compressible and expandable annular stent or frame 234 and one or more leaflets 236 supported in the frame to regulate the flow of blood through the valve in one direction. The prosthetic valves can be self-expandable or plastically-expandable. Some examples of prosthetic valves that can be used are disclosed in, for example, U.S. Pat. Nos. 7,993,394; 7,393,360; and 8,652,202, and U.S. Publication No. 2012/0123529, which are incorporated herein by reference.

In some embodiments, it may be desirable to implant a prosthetic valve in only one of the orifices 26, 28 while leaving the other orifice 26, 28 without a prosthetic valve.

Any of the embodiments described herein can be used with a previously implanted fixation device 10, or a newly implanted fixation device 10. For example, any of the embodiments described herein can be implanted in a heart in which a fixation device 10 had been implanted years, months, weeks, or days earlier.

In other cases, any of the embodiments described herein can be implanted in a heart immediately following the implantation of a fixation device 10. Thus, any of the methods for treating an insufficient heart valve disclosed herein can include the step of implanting a fixation device 10 in the native heart valve.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An implantable device for remodeling a native mitral valve having two native leaflets and a fixation device or an Alfieri stitch secured to respective free edges of the leaflets, the implantable device configured to be coupled to the fixation device or Alfieri stitch and apply a remodeling force to the native mitral valve that draws the leaflets toward each other to promote coaptation of the leaflets.

2. The implantable device of claim 1, wherein the remodeling force applied by the implantable device draws the leaflets and the chordae tendineae closer toward the left atrium.

3. The implantable device of claim 2, wherein the device comprises a tension member configured to be coupled to the fixation device or Alfieri stitch and an anchor member connected to the tension member, the anchor member being configured to be engage tissue in the left atrium, the intra-atrial septum, and/or a pulmonary vein.

4. The implantable device of claim 3, wherein the anchor member comprises an expandable stent sized to be implanted within a pulmonary vein.

5. The implantable device of claim 4, wherein the stent includes an eyelet through which the tension member can extend.

6. The implantable device of claim 3, wherein the anchor member comprises a first anchor portion and a second anchor portion, the first anchor portion being configured to engage the intra-atrial septum in the left atrium and the second anchor portion being configured to engage the intra-atrial septum in the right atrium.

7. The implantable device of claim 3, wherein the tension member comprises a suture.

8. The implantable device of claim 1, wherein the remodeling force applied by the implantable device causes the leaflets to be twisted about an axis extending parallel to the flow of blood from the left atrium to the left ventricle.

9. The implantable device of claim 8, wherein the device is configured to be anchored to tissue in the left ventricle or the left atrium.

10. A method for treating a native mitral valve of a heart, the mitral valve having two native leaflets and a fixation device or an Alfieri stitch secured to respective free edges of the leaflets, the method comprising:
delivering a remodeling device into the heart;
coupling the remodeling device to the fixation device or Alfieri stitch; and
applying a remodeling force to the native mitral valve via the remodeling device, the remodeling force drawing the leaflets toward each other to promote coaptation of the leaflets.

11. The method of claim 10, further comprising anchoring an anchor member of the remodeling device to tissue in or adjacent the heart to maintain the remodeling force on the native mitral valve.

12. The method of claim 10, wherein the remodeling force extends in a direction toward the left atrium and draws the leaflets and the chordae tendineae closer to the left atrium.

13. The method of claims 10, the remodeling device comprises a tension member that is coupled to the fixation device or Alfieri stitch and is held in tension by an anchor member of the remodeling device that is anchored to tissue in or adjacent the left atrium.

14. The method of claim 13, wherein the tension member forms a loop around the fixation device or Alfieri stitch and has two ends connected to the anchor member.

15. The method of claim 13, wherein the anchor member is anchored to the intra-atrial septum.

16. The method of claim 13, wherein the anchor member comprises a stent implanted in a pulmonary vein.

17. The method of claim 10, wherein the remodeling force causes the leaflets to be twisted about an axis extending parallel to the flow of blood from the left atrium to the left ventricle.

18. A method for treating a native mitral valve of a heart, the mitral valve having two native leaflets and a fixation device or an Alfieri stitch secured to respective free edges of the leaflets, the method comprising:
coupling a docking member to the fixation device or Alfieri stitch; and
deploying a prosthetic valve within the docking member.

19. The method of claim 18, wherein coupling a docking member to the fixation device or Alfieri stitch comprises deploying a rail around the fixation device or Alfieri stitch and advancing the docking member along the rail to a location adjacent the native mitral valve within the left atrium.

20. The method of claim 18, wherein the docking member comprises a radially extending flange that forms a seal against the inner surface of the left atrium.

21. The method of claim 18, wherein the prosthetic valve is delivered into the heart in a radially compressed state by a delivery catheter and then radially expanded to an expanded state within the docking member.

22. A method for treating a native mitral valve of a heart, the mitral valve having two native leaflets and a fixation device or an Alfieri stitch secured to the leaflets at a location between the commissures so as to define two orifices between the leaflets separated by the fixation device or Alfieri stitch, the method comprising:
implanting a prosthetic valve within one of the orifices.

23. The method of claim 22, further comprising implanting another prosthetic valve in the other orifice.

24. The method of claim 23, wherein the prosthetic valves are connected to each other by a connecting member.
